(19) United States
(12) Patent Application Publication
(10) Pub. No.: US 2009/0118677 A1
(43) Pub. Date: May 7, 2009

(54) EXCHANGE NEEDLE RETRACTABLE SAFETY SYRINGE

(75) Inventors: Graeme Francis Walton, New South Wales (AU); Allan Walsh, New South Wales (AU)

Correspondence Address: BROOKS KUSHMAN P.C., 1000 TOWN CENTER, TWENTY-SECOND FLOOR, SOUTHFIELD, MI, 48075, US

(73) Assignee: GLOBAL MEDISAFE HOLDINGS PTY LIMITED, The Hill, NSW (AU)

(21) Appl. No.: 11/718,638

(22) PCT Filed: Apr. 4, 2005
(86) PCT No.: PCT/AU2005/000491

§ 371 Date: Sep. 26, 2007

(30) Foreign Application Priority Data
Nov. 4, 2004 (AU) 2004906332

(51) Int. Cl. A61M 5/32

(52) U.S. Cl. 604/196

(57) ABSTRACT

A safety syringe comprising a syringe barrel (12), a plunger (13), a needle (14), a needle hub (15) integral with the needle, a needle gland (16) to accommodate the needle-and hub, and a needle cap (19) to protect the needle prior to use. The plunger has engagement means (25, 36) located on its end to engage with the needle hub in order to retract the needle from the gland and into the syringe barrel after the syringe has been used. The plunger is provided with a threaded split bush (28), which has a multi start thread. When the plunger is fully retracted with the needle attached, the plunger has one or more lugs (31) that come into contact with the threaded split bush so that the plunger is thereby locked. Hence the needle is locked in the barrel. The plunger may be snapped off retaining the needle safely within the syringe barrel.

### TECHNICAL FIELD

This invention relates to a safety syringe having a retractable needle for use in the medical or dental profession or in personal drug administration so that the physician, surgeon or other needle operator might be protected from injury by the needle after its use. In particular, it relates to safety syringes where there is a requirement to accommodate various sizes of needles or where a needle change is required between filling the syringe from a vial and giving an injection to the patient.

### BACKGROUND

The danger of injury and possible infection from the HIV or hepatitis B virus to medical practitioners using needles in the normal course of their business is well documented.

Furthermore, persons who are in the habit of administering drugs to themselves run a severe risk of contacting either of the specified viruses, or indeed contacting other viruses if a needle, once used, is reused in an unsterilised form. There are also added dangers where the needle in the syringe is required to be replaced.

There have been many proposals aimed at reducing the number of so-called needle-stick injuries and various attempts have been made to provide a safe system for disposal of such needles once used, but such prior proposals have had deficiencies.

### OBJECT OF THE INVENTION

It is an object of the present invention to provide a syringe having a retractable surgical needle which is able to accommodate various sizes of surgical needles or which allows a needle change between filling the syringe from a vial and giving the injection to the patient. It is also an object of the invention to provide a syringe which employs such a retractable needle in a sterile manner prior to use, and for permanently storing that surgical needle, once used, in a substantially safe manner.

At the very least the invention provides an alternate means for providing an exchange needle and/or accommodating the needle of a used syringe to protect against accidental injury arising from unwanted contact with the exposed needle once the syringe has been used.

Although the following description generally refers to a syringe of conventional size, no such limitation is intended thereby, and reference to a syringe is meant to encompass any other needle/syringe combination including slimline syringes, where, by suitable adaptation, the invention may also be usefully applied.

### DISCLOSURE OF THE INVENTION

The invention in one broad form provides a safety syringe incorporating a retractable exchange needle has a syringe barrel which has a plunger slideably located therein, with piston means located towards the end of the plunger remote from the end of the barrel from which the plunger extends, the syringe barrel further having means to removably attach a needle assembly at the end of the barrel opposite that from which the plunger extends, the needle assembly comprising a needle, a needle hub integral with the needle, a needle gland to accommodate the needle and hub, and a needle cap to protect the needle prior to use; wherein the plunger has engagement means located on its end to engage with the needle hub in order to retract the needle from the gland and into the syringe barrel after the syringe has been used, and wherein the plunger is provided with a threaded split bush, characterised in that the inside of the threaded spilt bush has multi start thread which when the plunger is fully retracted with the needle attached, the plunger has one or more lugs that come into contact with the threaded split bush which thereby locks the plunger and hence the needle in the barrel such that the plunger may be snapped off retaining the needle safely within the syringe barrel.

Preferably the gland is fitted to the end of the barrel by threaded means.

Prior to use, the needle cap protects the needle from damage and also acts as a safety cap for the medical staff using the syringe preventing needle stick injuries. The presence of the needle cap also means that the needle assembly may be conveniently changed without risk of needle stick injury which might occur if the needle were unprotected.

The needle gland is preferably held in the needle cap by interference fit. Preferably there are matching splines on the outside of the needle gland that match with splines on the inside of needle cap. The needle hub is pushed into the needle gland and is held in position by a matching ring that is an interference fit. A sealing fit is achieved by this method.

Following assembly, ie after the needle gland is assembled with the needle inside and the protective needle cap is fitted, the assembly is screwed inside the end of the barrel. A sealing point is secured by means of matching tapers between the needle gland and the inside of the threaded barrel.

The barrel of the syringe is preferably of clear plastic and is graduated to read fluid level. After the needle gland with the needle hub fitted therein, is screwed into the barrel and the seal is reached, the needle cap is removed from the assembly making the needle ready for use. Various size needle assemblies can thus be fitted to the barrel.

The piston is preferably made of synthetic rubber and is fitted to the plunger so as to seal by way of an interference fit between the plunger and the barrel, thereby allowing the fluid to be extruded from the syringe, via the needle, in the usual manner.

Preferably the plunger has built into its end a clip to engage with the needle hub after the plunger has expelled the fluid from the syringe, ie after completing an injection. When the two mating parts are pushed together, the plunger is then pulled back causing the used needle hub inclusive of the steel needle to come out of the needle gland. The needle is now connected to the end of the plunger and can be withdrawn into the body of the syringe. Preferably the plunger has a snap point created by way of reduced diameter near the piston. When the plunger is snapped off, the syringe is rendered inoperative.

The split threaded bush is located around the body of the plunger and pushed into the barrel end and is held in position by way of an interference fit and a matching groove. Preferably, inside the split bush is a multi-start thread which when the plunger is fully retracted with the needle attached, the plunger has one or more lugs that come into contact with the threaded split bush. When the threaded split bush is thus engaged by the plunger, the plunger is locked to the end of the barrel. The plunger can then be snapped off. The snap point on the plunger is preferably designed to break off clean and flat with the top of the barrel making it hard to remove the needle from the barrel.

Optionally the method of locking the needle hub to the plunger may be in reverse, ie the female half can be in the end of the needle hub and the male locking protrusion is on the plunger. Also the protrusion on the end of the plunger (male fitting) can incorporate a stainless steel clip having a barb which locks in the needle hub when fully engaged after injection of medical fluid.

In this embodiment, the stainless steel pressed clip is made from sheet and pushes over the male protrusion. The stainless steel clip is made in the form of a “C” section that clamps around the male protrusion approximately 5 mm long with a barb being formed on one side to interact with the inner wall of the needle hub at the completion of injection. After the needle is removed from the patient, the plunger is pulled back, the stainless steel barb on the plunger having locked into the needle hub causes the needle hub and thus the needle to retract into the body of the barrel rendering the needle safely stored from human harm. The separation of the needle from the plunger cannot be achieved and when the plunger is retracted and locked into the threaded split bush and the plunger is snapped off, the syringe is locked and is unable to be used again. It will be appreciated that the protrusion on the plunger has an interference fit to the inside of the needle hub and this makes it obvious when filling the syringe prior to injection at which point the plunger “bottoms” in the syringe barrel. However, after injection a more powerful stroke is required to overcome the initial resistance in order to actually lock the needle hub onto the plunger.

The advantages of the invention lie in:

1. The manual retraction of the needle preferably using a stainless clip.

2. The twist lock split bush locking the plunger in the barrel prior to snapping off the plunger.

3. Using the above principals, the plunger can be retracted without fluid splash caused by auto retract syringes.

4. The screw in needle assembly allows for variance in needle gauge and/or a needle exchange can be made after the syringe has been filled from a vial prior to injection.

### BRIEF DESCRIPTION OF THE DRAWINGS

The invention may be better understood from the following non-limiting description of a preferred embodiment, in which:

FIG. 1 is a cross sectional view of an assembled syringe according to one aspect of the invention in which the end of the plunger has a female fitting,

FIG. 2 is a cross sectional exploded view of a components utilised in the syringe Assembly of FIG. 1,

FIG. 3 is a cross sectional view of a syringe according to a second embodiment of the invention, where the plunger has a male fitting,

FIG. 4 is a detailed side view of a plunger and needle for use in a syringe according to the second embodiment of FIG. 3,

FIG. 5 shows the detail of the stainless steel clip used on the plunger of FIGS. 3 and 4, and

FIG. 6 is a perspective view of a split bush for use with the syringe of either embodiment.

### DETAILED DESCRIPTION OF THE DRAWINGS

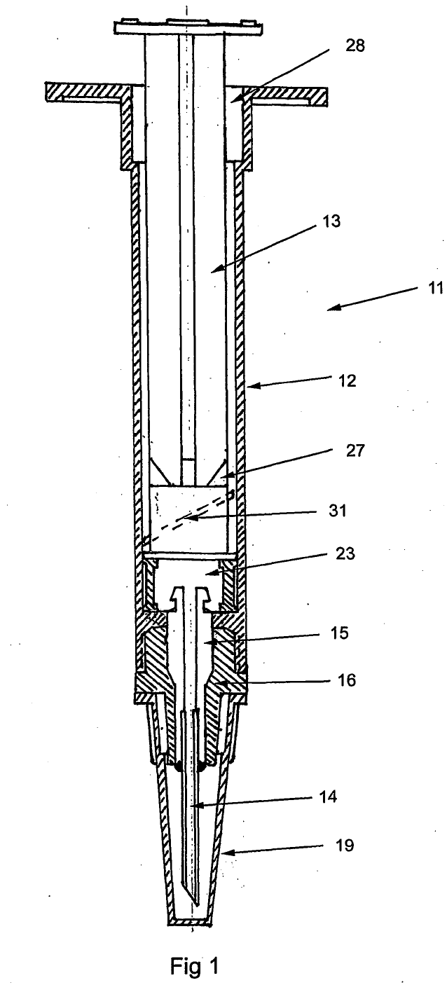

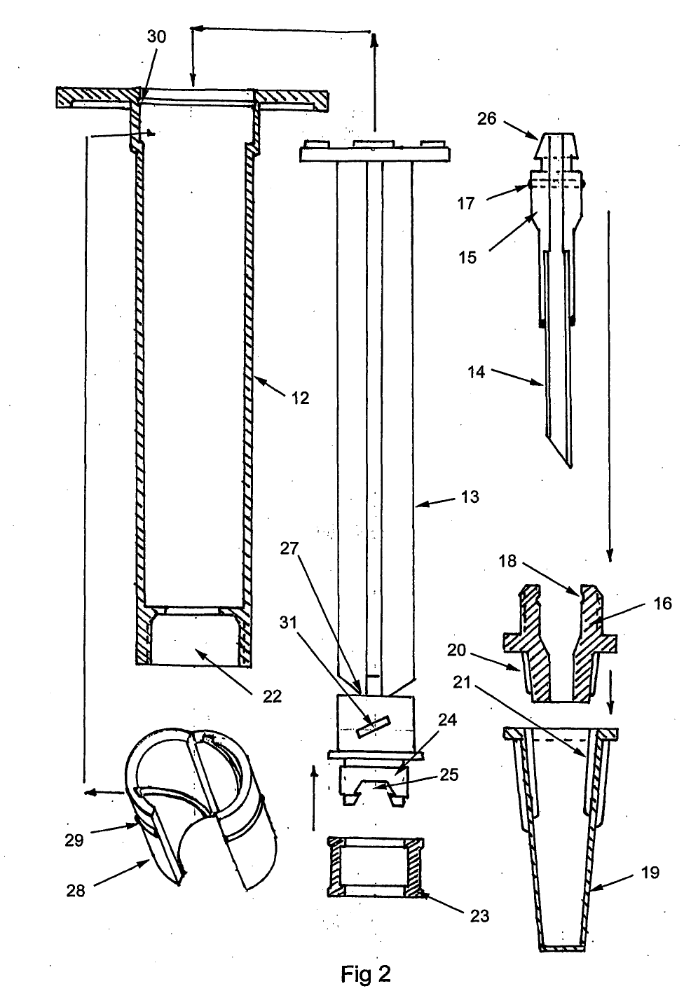

Referring generally to FIGS. 1 and 2 there is shown a syringe and needle combination generally referenced 11, which comprises a syringe barrel 12, a plunger 13 and a needle 14. The needle 14 is provided with a needle hub 15 at its upper end.

The needle hub 15 is pushed into the needle gland 16 and is held in position by a ridge 17 about the periphery of the hub 15 which mates with a corresponding recess 18 about the inner periphery of the gland 16 in an interference fit. A sealing fit is thus achieved by this method.

A needle cap 19 protects the needle from damage and also acts a safety cap for the medical staff using the syringe preventing needle stick injuries.

The needle gland 16 is held in the needle cap 19 by interference fit. There are matching splines 20 on the outside of the needle gland 16 that match with complementary recesses 21 on the inside of needle cap 19. After the needle gland 16 is assembled with the needle 14 inside, and the cap 19 fitted, the assembly 14,16,19 is screwed inside the end 22 of the barrel 12. A sealing point is achieved by means of matching tapers between the needle gland 16 and the inside of the threaded portion 22 of barrel 12.

The barrel 12 is of clear plastic and is graduated to read fluid level. After the needle assembly 14,16,19 is screwed into the barrel 12 by virtue of gland 16 being screwed therein, and the seal is reached, the needle cap 19 is removed making the needle 14 ready for use. Various size needle assemblies 14,16,19 can thus be fitted to the barrel 12.

The plunger 13 is provided with a piston 23 of synthetic rubber which is fitted to the plunger 13 and seals by way of an interference fit between the plunger 13 and the barrel 12 allowing the fluid to be extruded from the syringe 11 in the usual manner.

The plunger 13 has built into its end 24 a clip means 25 which in this embodiment is a female clip arrangement to engage with a correspondingly designed male portion 26 on the needle hub 15 after the plunger 13 has expelled the fluid (not shown) from the syringe 11. When the two mating parts 25,26 are pushed together, the plunger 13 is pulled back causing the used needle hub 15 inclusive of the steel needle 14 to come out of the needle gland 16. The needle 14 is now connected to the end of the plunger 13 and can be withdrawn into the body 12 of the syringe 11. The plunger 13 has a snap point 27 by way of reduced diameter near the piston 23. When snapped off, the syringe 11 is thus rendered inoperative.

A split threaded bush 28 is located about the body of the plunger 13 and pushed into the end of the barrel 12 and is held in position by way of an interference fit from a ridge 29 on the bush and a matching groove 30 in the barrel 12. Inside the split bush 28 is a multi-start thread. When the plunger 13 is fully retracted with the needle 14 attached, the two lugs 31 located on the plunger 13 come into contact with the threaded split bush 28. When the threaded split bush 28 is thus engaged by the plunger 13, the plunger 13 is thereby locked to the end of the barrel 12. The plunger 13 can now be snapped off at the snap point 27, which has been designed to break off clean and flat with the top of the barrel 12 thus making it hard to remove the needle 14 from the barrel 12.

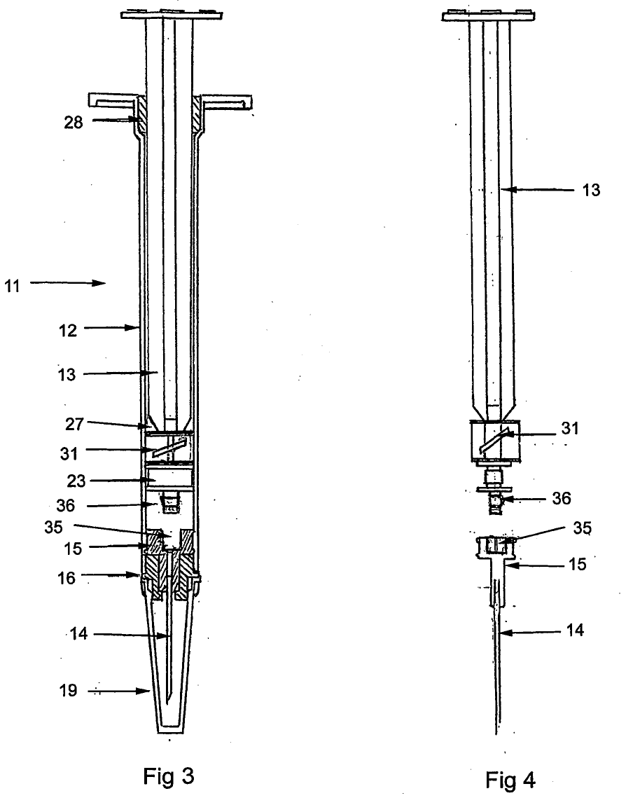

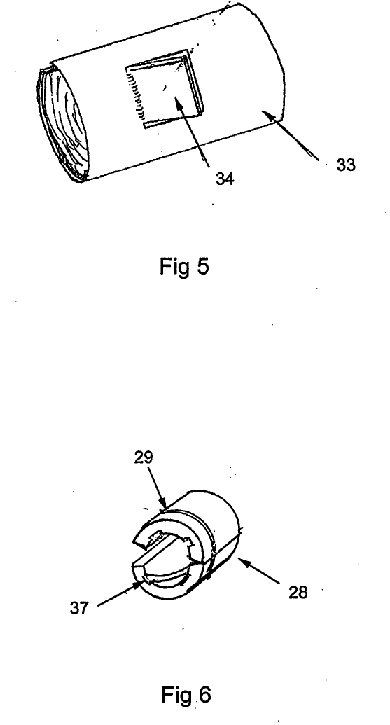

Turning to FIGS. 3, 4 and 5, there is shown a syringe 11 having plunger 13 of an alternate embodiment in which like components to those illustrated in FIGS. 1 and 2 are referenced with the same numerals. In this case however, the method of locking the needle hub 15 to the plunger 13 is opposite in that the female portion 36 is located in the end of the needle hub 15, whilst the male locking protrusion 36 is located at end of the plunger 13. In this case, the protrusion 36 on the end of the plunger 13 is fitted with a stainless steel clip 33 (shown in detail in FIG. 5) having a barb 34 which locks in the needle hub 15 when fully engaged after injection of medical fluid. The stainless steel pressed clip 33 is made from sheet and pushes over the male protrusion 32. The stainless steel clip is made in the form of a “C” section that clamps around the male protrusion 32 approximately 5 mm long with the barb 34 being formed on one side to interact with the inner wall of the needle hub 15 at the completion of injection. Barb 34 may be square in shape as shown, or for example triangular in shape ending in a point for greater biting capacity.

When the plunger 13 is pulled back, after the needle 14 is removed from the patient, the stainless steel barb 34 on the plunger 13 locks into the needle hub 15 and retracts the needle hub 15 and thus the needle 14 into the body 12 of the syringe 11 rendering the needle 14 safely stored from human harm. The separation of needle 14 from the plunger 13 cannot be achieved and when the plunger 13 is retracted and locked into the threaded split bush 28 (as in the earlier FIGS. 1 and 2) and the plunger 13 is snapped off, the syringe 11 is then locked and is not able to be used again.

FIG. 6 shows an alternative split bush 28 for use in either embodiment of FIGS. 1 ot 2 or FIGS. 3 to 5, where there is utilised a four start twist thread 37.

It will be appreciated by those skilled in the art that many modifications and variations may be made to the embodiments described herein without departing from the spirit or scope of the invention.

Throughout the specification the word “comprise” and its derivatives are intended to have an inclusive rather than exclusive meaning unless the context requires otherwise.

1. A safety syringe having a syringe barrel incorporating a retractable exchange needle has a plunger slideably located therein, with piston means located towards the end of the plunger remote from the end of the barrel from which the plunger extends, the syringe barrel further having means to removably attach a needle assembly at the end of the barrel opposite that from which the plunger extends, the needle assembly comprising a needle, a needle hub integral with the needle, a needle gland to accommodate the needle and hub, and a needle cap to protect the needle prior to use; wherein the plunger has engagement means located on its end to engage with the needle hub in order to retract the needle from the gland and into the syringe barrel after the syringe has been used, and wherein the plunger is provided with a threaded split bush, characterised in that the inside of the threaded spilt bush has multi start thread which when the plunger is fully retracted with the needle attached, the plunger has one or more lugs that come into contact with the threaded split bush which thereby locks the plunger and hence the needle in the barrel such that the plunger may be snapped off retaining the needle safely within the syringe barrel.

2. A safety syringe according to claim 1 wherein the gland is fitted to the end of the barrel by threaded means.

3. A safety syringe according to claim 1 wherein prior to use, the needle cap protects the needle from damage and also acts as a safety cap for the medical staff using the syringe preventing needle stick injuries.

4. A safety syringe according to claim 1 in which the needle gland is held in the needle cap by interference fit.

5. A safety syringe according to claim 4 in which there are matching splines on the outside of the needle gland that match with splines on the inside of needle cap.

6. A safety syringe according to claim 1 in which the needle hub is pushed into the needle gland and is held in position by an interference fit.

7. A safety syringe according to claim 6 wherein following assembly of the needle in the needle gland and the protective needle cap is fitted, the needle assembly is screwed inside the end of the barrel.

8. A safety syringe according to claim 7 wherein a sealing point is secured by means of matching tapers between the needle gland and the inside of the threaded barrel.

9. A safety syringe according to claim 7, wherein after the needle assembly is screwed into the barrel and the seal is reached, the needle cap is removed from the assembly making the needle ready for use.

10. A safety syringe according to claim 9 wherein various size needle assemblies are able to be fitted to the barrel of the syringe.

11. A safety syringe according to claim 1 wherein the barrel of the syringe is preferably of clear plastic and is graduated to read fluid level.

12. A safety syringe according to claim 1 in which the piston is made of synthetic rubber and is fitted to the plunger so as to seal by way of an interference fit between the plunger and the barrel, thereby allowing the fluid to be extruded from the syringe, via the needle, in the usual manner.

13. A safety syringe according to claim 1 in which the plunger has built into its end a clip to engage with the needle hub after the plunger has expelled the fluid from the syringe, so that when the two mating parts are pushed together, the plunger is then pulled back causing the used needle hub inclusive of the steel needle to come out of the needle gland and be withdrawn into the body of the syringe.

14. A safety syringe according to claim 1 in which the plunger has a snap point created by way of reduced diameter near the piston, so that when the plunger is snapped off, the syringe is rendered inoperative.

15. A safety syringe according to claim 1 in which the split threaded bush is located around the body of the plunger and pushed into the barrel end and is held in position by way of an interference fit.

16. A safety syringe according to claim 15 in which the split bush has a multi-start thread therein, so that when the plunger is fully retracted with the needle attached, the plunger has one or more lugs that come into contact with the threaded split bush and the threaded split bush is thus engaged by the plunger and the plunger is thereby locked to the end of the barrel.

17. A safety syringe according to claim 16 in which the plunger is snapped off to render the syringe inoperative.

18. A safety syringe according to claim 1 in which the method of locking the needle hub to the plunger is by means of a female portion located on the needle hub and a male protrusion located on the end of the plunger.

19. A safety syringe according to claim 18 in which the male protrusion on the plunger incorporates a stainless steel clip having a barb which locks in the needle hub when fully engaged after injection of medical fluid.

20. A safety syringe according to claim 19 in which the stainless steel clip is made from pressed sheet and is pushed over the male protrusion, the stainless steel clip being made in the form of a “C” section that clamps around the male protrusion and is provided with a barb formed on one side to interact with the inner wall of the needle hub at the completion of injection.
